(19) United States
(12) Patent Application Publication
(10) Pub. No.: US 2026/0340531 A1
(43) Pub. Date: Oct. 29, 2026

(54) MATCHING ENGINE AND SYSTEM FOR LABORATORY COMPUTATION LOAD BALANCING

(71) Applicant: WELAB TECHNOLOGIES INC., Mississauga (CA)

(72) Inventor: Doughan TURK, Mississauga (CA)

(21) Appl. No.: 19/114,333

(22) PCT Filed: Oct. 18, 2023
(86) PCT No.: PCT/IB2023/060535

§ 371 Date: Mar. 21, 2025

Related U.S. Application Data
(60) Provisional application No. 63/419,084, filed on Oct. 25, 2022.

(51) Int. Cl. G06Q 10/0631; G06Q 10/047; G16H 40/20; H04W 4/029

(52) U.S. Cl. CPC G06Q 10/063114; G06Q 10/047; G16H 40/20; H04W 4/029

(57) ABSTRACT

The specification provides, amongst other things, a method and system for computational load balancing through a matching engine. The method and system can involve the receipt of requisition messages and the acquisition of geolocation data from primary devices, mobile devices, and computation devices. The method and system can construct a geospatial routemap for mobile devices based on these data points, detailing their journey from their current location, through primary device locations, and onto computation device locations.

### CROSS-REFERENCE TO RELATED APPLICATION(S)

This application claims the benefit of U.S. Provisional Patent Application No. 63/419,084, filed Oct. 25, 2022, entitled “LABORATORY SERVICES DEPLOYMENT SYSTEM AND MATCHING ENGINE”; the entire contents of which are incorporated herein by reference.

### BACKGROUND

Health care services are an important component of maintaining over-all public health and a well-functioning society. The recent COVID-19 pandemic highlighted how a medical care system can be brought close to a breaking point when an unexpected public health emergency arises.

### SUMMARY

An aspect of the present specification provides a method for deploying laboratory services comprising receiving requisitions, receiving patient geodata, receiving technician geodata, generating routes for technicians to follow to locate patients, and confirming service delivery.

An aspect of the specification provides a matching engine for computational load balancing including a network interface, a processor and a memory for storing programming instructions for configuring the processor to: receive at least one laboratory requisition electronic message; receive at least one primary geolocation dataset of at least one target location of at least one primary electronic device respective to each message; receive at least one mobile geolocation dataset of at least one location of at least one mobile electronic device within a geographic range proximal to at least one primary electronic devices of the primary geolocation dataset; receive at least one secondary geolocation dataset of at least one final location of at least one computation device within a geographic range proximal to the primary geolocation dataset; generate a geospatial electronic routemap for each mobile electronic device including travel from i) an origin within the mobile geolocation dataset, to ii) the at least one target location of one or more primary electronic devices, and then to iii) the at least one final location of the at least one computation device; generate expected electronic states for each stage of the electronic routemap; assign tolerances to the expected electronic states representing expected time-frames associated with each of the states; receive real-time to updates of actual electronic states of each mobile electronic device and comparing to the expected electronic states; and, updating the geographic electronic routemap for at least one of the mobile electronic devices if the actual electronic states are outside the tolerances of the expected electronic states.

An aspect of the specification provides a matching engine, wherein the tolerances are based at least in part on a time-frame for potential sample degradation during travel from the target location of a respective primary device to an assigned computation device location.

An aspect of the specification provides a matching engine or claim 12, wherein the tolerances are based at least in part on arrival time windows at the target locations of the primary devices.

An aspect of the specification provides a matching engine wherein the tolerances are based at least in part on reducing the deployment of mobile devices of laboratory technicians.

An aspect of the specification provides a matching engine wherein the tolerances are based at least in part on reducing a carbon footprint of vehicles associated with the mobile devices.

An aspect of the specification provides a matching engine wherein the time-frame tolerances are based at least in part maximizing a number of primary devices assigned per mobile device.

An aspect of the specification provides a matching engine, wherein criteria for adjusting include real-time monitoring of technician mobile device availability and reallocation of technician mobile device to uphold primary device appointment commitments and maintain sample integrity.

An aspect of the specification provides a matching engine, wherein the adjusting includes assessing if scheduling adjustments can be implemented without increasing the likelihood of missed primary device appointment commitments.

An aspect of the specification provides a matching engine, further including machine learning to evaluate the impact of scheduled routes or changes to said routes on tolerance violations, and adjust criteria for the generating and the adjusting based on the evaluation results.

An aspect of the specification provides a matching engine wherein the predicted states can further include one or more of i) parking a vehicle; ii) administering the test in the requisition; iii) waiting for processing of test results.

An aspect of the specification provides a method for computational load balancing by a matching engine including: receiving at least one laboratory requisition message; receiving primary geolocation data of target locations of primary devices related to each message; receiving mobile geolocation data of mobile devices proximal to the primary devices; receiving secondary geolocation data of computation device locations near the primary locations; generating a geospatial routemap for each mobile device respective to each said message including travel from: i) a starting point in the mobile geolocation data, to ii) target locations of primary devices, and then to iii) computation device locations; generating predicted electronic states for each routemap stage; assigning tolerances for each of the states; monitoring real-time electronic state updates of mobile devices and compare with the predicted states; and, adjusting the routemap for one or more of the mobile devices if any real-time state diverges from the tolerances.

An aspect of the specification provides a method, wherein the tolerances are based at least in part on a time-frame for potential sample degradation during travel from the target location of a respective primary device to an assigned computation device location.

An aspect of the specification provides a method or claim 12, wherein the tolerances are based at least in part on arrival time windows at the target locations of the primary devices.

An aspect of the specification provides a method wherein the tolerances are based at least in part on reducing the deployment of mobile devices of laboratory technicians.

An aspect of the specification provides a method wherein the tolerances are based at least in part on reducing a carbon footprint of vehicles associated with the mobile devices.

An aspect of the specification provides a method wherein the time-frame tolerances are based at least in part maximizing a number of primary devices assigned per mobile device.

An aspect of the specification provides a method, wherein criteria for adjusting include real-time monitoring of technician mobile device availability and reallocation of technician mobile device to uphold primary device appointment commitments and maintain sample integrity.

An aspect of the specification provides a method, wherein the adjusting includes assessing if scheduling adjustments can be implemented without increasing the likelihood of missed primary device appointment commitments.

An aspect of the specification provides a method, further including machine learning to evaluate the impact of scheduled routes or changes to said routes on tolerance violations, and adjust criteria for the generating and the adjusting based on the evaluation results.

An aspect of the specification provides a method wherein the predicted states can further include one or more of i) parking a vehicle; ii) administering the test in the requisition; iii) waiting for processing of test results

### BRIEF DESCRIPTION OF THE FIGURES

FIG. 1 is a schematic diagram of a system for computational load balancing of deployment of laboratory services.

FIG. 2 is a block diagram of the server of FIG. 1.

FIG. 3 shows a flowchart depicting a method for computational balancing of deployment of laboratory services.

FIG. 4 shows a flowchart depicting a method for routing deployed laboratory services.

FIG. 5 shows a flowchart depicting a method for delivery of the laboratory services.

FIG. 6 shows a flowchart depicting a method for computational balancing of deployment of laboratory services.

FIG. 7 is an assistive visual aid to appreciate technical performance of the method of FIG. 6 and/or related methods.

FIG. 8 is an assistive visual aid to appreciate technical performance of the method of FIG. 6 and/or related methods.

FIG. 9 is an assistive visual aid to appreciate technical performance of the method of FIG. 6 and/or related methods.

FIG. 10 is an assistive visual aid to appreciate technical performance of the method of FIG. 6 and/or related methods.

FIG. 11 is an assistive visual aid to appreciate technical performance of the method of FIG. 6 and/or related methods.

### DETAILED DESCRIPTION

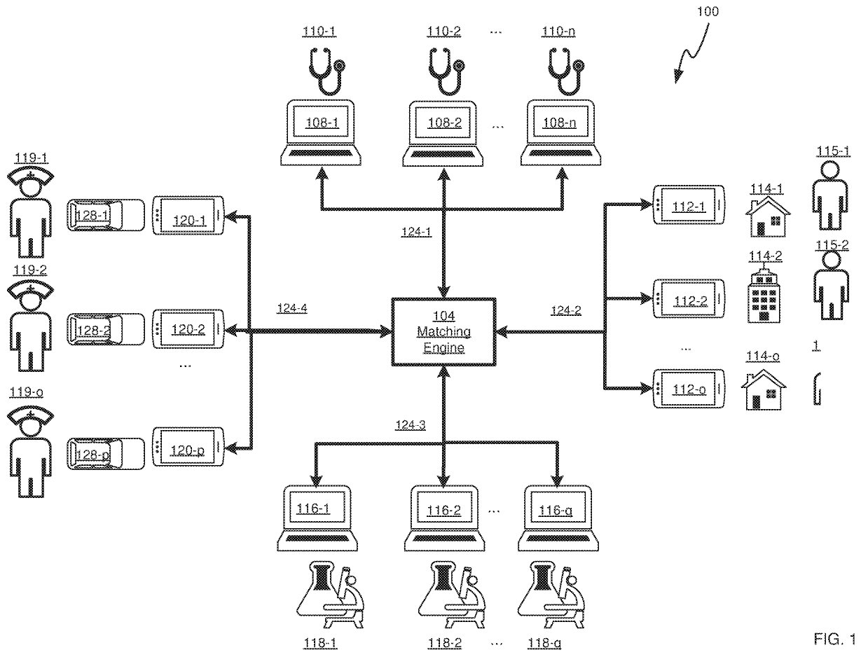

FIG. 1 shows a system for computational balancing of deployment of laboratory services indicated generally at 100. The locus of system 100 is a matching engine 104 that interconnects a plurality of requisitioner devices 108, a plurality of patient devices 112, a plurality of laboratory devices 116 and a plurality of technician devices 120.

A person skilled in the art will observe that in FIG. 1, requisitioner devices 108 are labelled individually as requisitioner device 108-1, 108-2 . . . 108-n. Collectively, these are referred to as devices 108 and generically as device 108. This nomenclature is used elsewhere herein, including for patient devices 112, laboratory devices 116 and technician devices 120.

In system 100, the links 124 to matching engine 104 can be implemented with any network infrastructure, but typically would be implemented over the Internet, utilizing encryption over wired and/or wireless technologies as the context requires.

Each requisitioner device 108 can be based on a laptop computer, desktop computer, mobile phone or the like. Requisitioner devices 108 are typically operated by any requisitioning entity 110 that provides medical care or other services and are legally authorized to make such requisitions for individuals or patients, such as physicians, nurses, employers, insurance underwriter or infectious disease testing. Typically, such a requisitioning entity would have authorization from the individual or patient. Electronic requisition messages can be received at matching engine 104 for further processing, as will be discussed in greater detail below.

Each patient device 112 can be based on a laptop computer, desktop computer, mobile phone or the like. Patient devices 112 are operated by any patient that would be receiving any service subject to a requisition. Patient devices 112, as will be explained in greater detail below, are typically associated with a fixed location 114 such as a home or a place of business, where the patient 115 would be expecting to receive the services associated with the electronic requisition message. Patient devices 112 generate scheduling, workflow, location and unique identity tracking associated with the delivery of requisitioned services, as will be discussed in greater detail below. (Note that in variants, an individual or patient may requisition services directly, such as infectious disease testing or bloodwork relating to monitoring a condition that does not require a health care professional, thereby obviating the need for separate requisitioner devices 108 to be used to send such an electronic requisition message to matching engine 104.)

Each laboratory device 116 can be based on a laptop computer, desktop computer, mobile phone or the like. Laboratory devices are operated by individual laboratories that complete the analysis of tests performed on patients operating devices 112. Laboratory devices 116, as will be explained in greater detail below, are typically associated with a laboratory 118 that performs the analysis. The results of such analyses are entered a respective device 116 and forwarded to matching engine 104 for further processing, such as data aggregation and meta-analysis, delivery of results to a respective patient device 112 and/or to a respective requisitioner device 108. The laboratory devices 116 are thus computation devices that can process and/or store and/or deliver lab test results to devices 108 and/or device 112.

Each technician device 120 is typically based on a mobile computing device such as a tablet computer or mobile telephone. Accordingly, the respective links 124-4 (or portions thereof) that connect to technician devices 120 are typically wireless. Technician devices 120 are operated by mobile laboratory technicians 119 such as nurses or phlebotomists or the like who are trained to fulfill the collection of testing samples from the patients who operate patient devices 112. Such laboratory technicians are referred to as being “mobile” because they operate vehicles 128 that can transport the technicians to a fixed location 114 where a given patient, operating their respective device 112, is located. The vehicles 128 thus render the technician devices 120 as mobile devices. At arrival the relevant location 114, the technician can perform their sample collection with the patient. As will be explained in greater detail below, routes for a given vehicle 128, according to their travel proximity to one or more given fixed locations 114, can be generated by matching engine 104 to deploy laboratory technicians to provide services to a given patient.

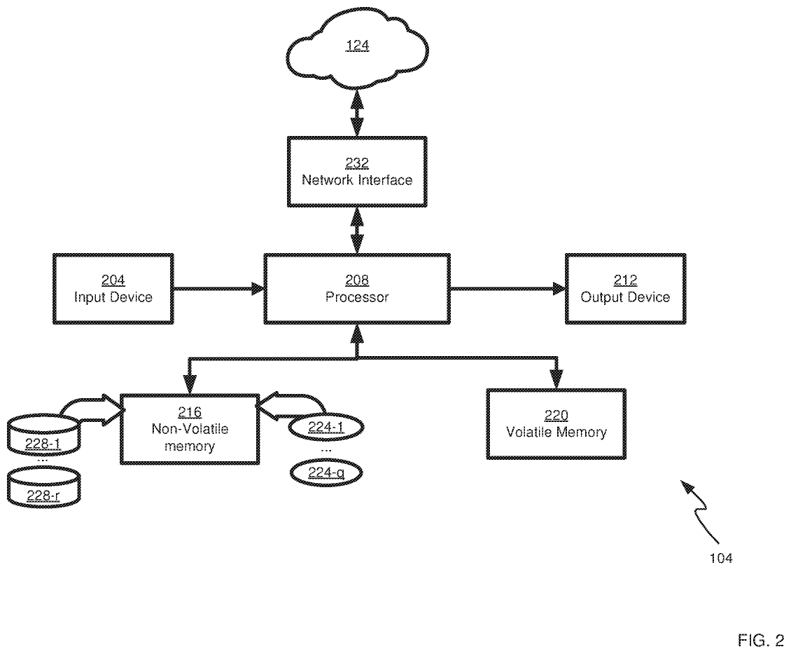

FIG. 2 shows a schematic diagram of a non-limiting example of internal components of matching engine 104. Matching engine 104 includes at least one input device 204. Input from device 204 is received at a processor 208 which in turn controls an output device 212. Input device 204 can be a traditional keyboard and/or mouse may be connected to provide physical input. Likewise output device 212 can be a display or audio speakers. In variants, additional and/or other input devices 204 or output devices 212 are contemplated or may be omitted altogether as the context requires. Matching engine 104 can be implemented in a data centre and/or on a Cloud computing platform such as Amazon webservices or Google Cloud, and/or as a plurality of mirrored instances for load balancing and/or massive scaling.

Processor 208 may be implemented as a plurality of processors or one or more multi-core processors. The processor 208 may be configured to execute different programing instructions responsive to the input received via the one or more input devices 204 and to control one or more output devices 212 to generate output on those devices.

To fulfill its programming functions, the processor 208 is configured to communicate with one or more memory units, including non-volatile memory 216 and volatile memory 220. Non-volatile memory 216 can be based on any persistent memory technology, such as an Erasable Electronic Programmable Read Only Memory (“EEPROM”), flash memory, solid-state hard disk (SSD), other type of hard-disk, or combinations of them. Non-volatile memory 216 may also be described as a non-transitory computer readable media. Also, more than one type of non-volatile memory 216 may be provided.

Volatile memory 220 is based on any random access memory (RAM) technology. For example, volatile memory 220 can be based on a Double Data Rate (DDR) Synchronous Dynamic Random-Access Memory (SDRAM). Other types of volatile memory 220 are contemplated.

Processor 208 also connects to links 124 (e.g. the Internet) via a network interface 232 which includes a buffer, a modulator/demodulator or MODEM. Network interface 232 can also be used to another computing device that has an input and output device, such as a keyboard, mouse and display, thereby obviating the need for input device 204 and/or output device 212 altogether.

Programming instructions in the form of applications 224 are typically maintained, persistently, in non-volatile memory 216 and used by the processor 208 which reads from and writes to volatile memory 220 during the execution of applications 224. One or more tables or databases 228 can also be maintained in non-volatile memory 216 for use by applications 224. The databases 228 can maintain various dataset such as geolocation datasets for requisitioner devices 108, patient devices 112, vehicles 128, and/or laboratory devices 116.

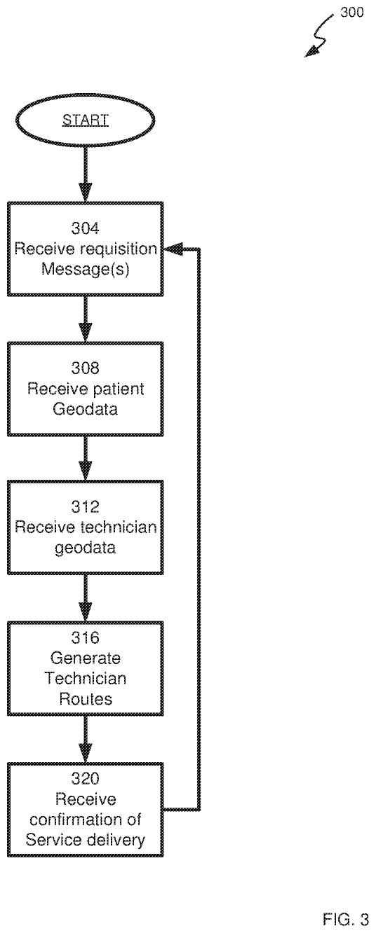

FIG. 3 shows a flowchart depicting a method for computational balancing of deployment of laboratory services indicated generally at 300. Method 300 can be implemented on matching engine 104 within the framework of system 100. Thus, persons skilled in the art may choose to implement method 300 on matching engine 104, system 100 or variants thereon, or with certain blocks omitted, performed in parallel or in a different order than shown. Method 300 can thus also be varied. However, for purposes of explanation, method 300 as per the flow chart of FIG. 3 and will be described in relation to its performance on matching engine 104 within the framework of system 100 of FIG. 1.

Block 304 comprises receiving a requisition message. Such a requisition message can be generated at a requisitioner device 108 which sends the requisition message to requisitioner device 108 where it is received. In a contemplated scaled version of system 100, at least one requisition messages can be received from a plurality of the requisition devices 108 during a given time frame. To reiterate from above, such a requisition can include a request for sample collection be obtained from a given patient as issued by a medical professional or the like. Such a requisition message can be structured according to best practices and/or legal standards of the relevant jurisdiction's regulatory authorities. The point being, the format and structure of the requisition messages are not particularly limited, but in substance at least include a unique identifier for a patient and one more unique identifiers of samples to be collected from that patient. The unique identifier can be associated with account that is maintained by matching engine 104 that matches the unique identifier with a patient that owns or operates a given patient device 112. The patient can be required to establish such an account through an account creation program and interface hosted by matching engine 104, complete with appropriate security, authentication, and encryption protocols along with a unique identifier for the account that uniquely and securely identifies the patient. That unique identifier, in turn, can be used as part of the requisition messages generated by requisition devices 108. Alternatively, matching engine 104 can be given access to a secure account hosting service such as those operated by Google, Facebook or a health care insurance provider, in order to uniquely identify a given patient so that a requisition message can be unambiguously associated with that patient.

It will now also be apparent that the requisitioner operating a respective device 108 will also have its own unique account, authentication credentials and encryption protocols with matching engine 104.

By the same token, each laboratory operating a respective device 116 and each technician operating a respective device 128 and will have their own unique accounts, authentication credentials and encryption protocols with matching engine 104.

The requisition message can also include a structured format so that the specific sample collections being identified for collection are unambiguous, using unique identifier codes, either pre-existing or defined for system 100, for example, that are respective to a given sample collection and testing type. For example, one unique code can represent “Iron Level Blood Sample”, while another represents “Cholesterol Level Blood Sample”. Additional unique codes for every laboratory service can be provided. This structured format can improve over existing paper-based requisition systems which may be subject to fraud as certain additional out-of-scope testing services are written in by hand after the requisitioner has created and issued the requisition.

Thus a requisition message will include a requisitioner unique identifier, a patient unique identifier, and one or more unique identifiers for the relevant sample collection type and associated laboratory test.

Block 308 comprises receiving patient geolocation data. In system 100, block 308 contemplates that each patient will have authenticated a session with matching engine 104 using a respective patient device 112. That session additionally will include an identification of a respective fixed location 114 of patient device 112, either automatically generated using location technology maintained on the relevant patient device or manually entered. Matching engine 104 can thus begin ascertaining relevant locations 114 for patients that are subject to a given requisition message received at block 304.

Block 312 comprises receiving technician geolocation data. In system 100, block 312 contemplates that each technician will have an authenticated session with matching engine 104 using a respective technician device 120. That session additionally will include an identification of a location of each device 120, typically generated using location technology maintained on the relevant technician device 120. Matching engine 104 can thus begin ascertaining locations of devices 120 for their geographic travel proximity to locations 114 of patients from block 308.

Block 316 comprises generating technician routes. Block 316 is based on data received at block 308 and block 312. In substance, the technician routes indicate, for each technician device 120, an expected routing that vehicles 128 can follow to one more fixed locations 112. The algorithms to effect block 316 are not particularly limited, but certain prioritizations can be preferred and such prioritizations can provide certain advantages. Depending on the number of requisitions, patients and technicians, the technician routings can be extremely complex, and may be changed in real-time according to traffic and any other events that frustrate timing expectations that are established during an initial performance of the algorithm.

In a simple example, assume two requisition messages are received. A first requisition message associated with device 112-1 and a second requisition message associated with device 112-2. Now assume that device 120-1 is physically closer to device 112-1 than it is to device 112-2; and assume that device 120-2 is physically closer to device 112-2 than it is to device 112-1. Thus, the routes generated at block 316 will direct: a) the technician associated with device 120-1 to the location 114 of the patient associated with the device 112-1; the technician associated with device 120-2 to the location 114 of the patient associated with the device 112-2.

In more complex examples, each technician may be assigned a plurality of patient requisitions. Thus optimized travel routes to minimize the amount of travel time for each technician can be generated at block 316. Furthermore as new requisition messages arrive when a given technician is already in the middle of a route, the technician routing can be updated in real time to accommodate fulfilment of a given sample collection from a given patient whose location 114 of the relevant patient device 112 may already be conveniently located near a technician device 120 whose vehicle 128 is already near that fixed location 114. Accordingly, a route can be generated once at the beginning of a working period, or updated periodically, or in real time, to provide efficient sample collection when a given technician may already be conveniently located proximate to a given patient.

While not required, block 316 can additionally comprise ascertaining the relative locations of given laboratory devices 116 in relation to fixed locations 114, to also optimize the routes generated at block 316. Likewise, if certain collected samples require faster delivery to a given laboratory, due to the need for faster test results or to ensure the integrity of the collected sample, then the routes can be optimized for more rapid delivery of the samples to the laboratories. The fact that matching engine 104 maintains geolocation data for each patient device 112 each technician device 120, and each laboratory device 116 facilitates such optimization.

Block 320 comprises receiving confirmation of service delivery. A person of skill in the art will now appreciate that method 300 contemplates the management of a plurality of requisitions for a plurality of patients and fulfilled by a plurality of technicians and laboratories. Typically, a confirmation of service delivery will occur each time a technician completes a sample collection and/or leaves such a sample with a given laboratory. In the technological realm, system 100 is configured so that a given collection sample interaction has certain authentication protocols conducted between a respective technician device 120 and a respective patient device 112. It is thus contemplated that an assignment of a requisition message to a particular technician device 120 will include the generation of a unique identifier for that requisition. That requisition unique identifier will be issued to the technician device 120 and the respective patient device 112 that is also uniquely associated with the relevant requisition from block 304. An authentication protocol is thus contemplated between those devices, such as generating a bar code bearing the requisition unique identifier is generated on given technician device 120 and can be scanned by the patient using their patient device 112. A match between the requisition unique identifier on the technician device 120 and the patient device 112 will affirm that the collection process may proceed. The requisition unique identifier may also be associated with preprinted, or dynamically printed, barcode labels that can be applied to collection sample vessels, thereby ensuring correct association of a collection sample with taken from a patient.

Block 320 can be augmented to including stages such as: confirmation that the sample was collected; confirmation that the sample was delivered to the laboratory, and confirmation that the laboratory has completed the testing on the sample.

These are non-limiting examples of how confirmation of service delivery at block 320 can be implemented, with a person of skill in the art how the various nodes in system 100 can be programmed to cooperate with matching engine 104.

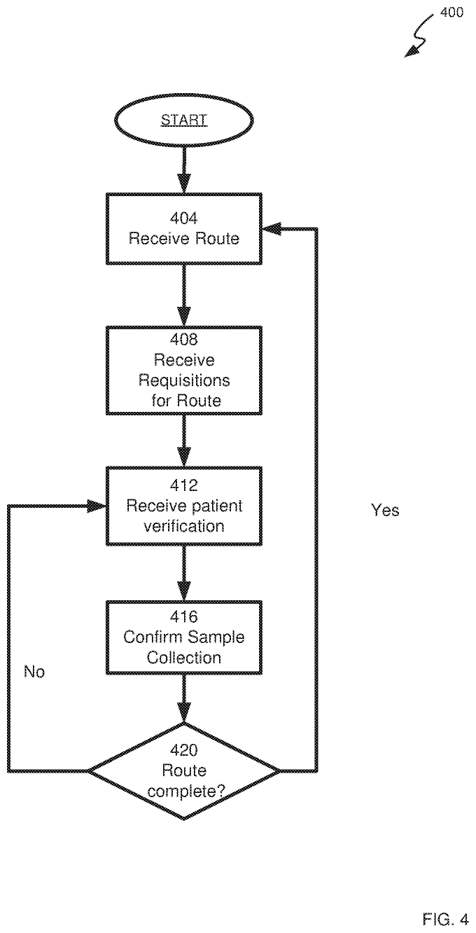

FIG. 4 shows a flowchart depicting a method of route fulfilment of a deployment of laboratory services indicated generally at 400. Method 400 can be implemented on a given technician device 128 within the framework of system 100. Thus, persons skilled in the art may choose to implement method 400 on one or more technician devices 104, system 100 or variants thereon, or with certain blocks omitted, performed in parallel or in a different order than shown. Method 400 can thus also be varied. However, for purposes of explanation, method 400 as per the flow chart of FIG. 4 and will be described in relation to its performance on technician device 128 within the framework of system 100 of FIG. 1.

Block 404 comprises receiving a route, and block 408 comprises receiving requisitions for the route. Such a route can be generated according to block 316 on method 300. Such a route is received on each technician device 128 to provide travel and, based on the requisitions from block 408, workflow instructions to a respective technician as to which fixed locations to attend 114, in which order, and which samples to collect from a given patient. The route and or requisitions may be updated in real time.

Block 412 comprises receiving patient verification. A technician operating device 128, when attending at a fixed location 114, can interact with a corresponding patient device 112 in order to confirm the patient's identity. One such approach is using the previously described bar code system associated with a unique requisition identifier.

Block 416 comprises confirmation of collection of a sample. Input can be received at technician device 120 either manually or involving scanning barcodes applied to collection sample vessels, where the barcodes include both the requisition unique identifier as well as an individual identification of the particular collected sample. All of this data can also be sent back to matching engine 112 as part of fulfillment of block 320.

Block 420 comprises determining if the route is complete. A “no” determination generates mapping instructions on technician device 120 to direct the technician to the next fixed location 114 and block 412 and block 416 are repeated for each fixed location 114 on the route. Block 420 can return to block 404 for new routes or route updates.

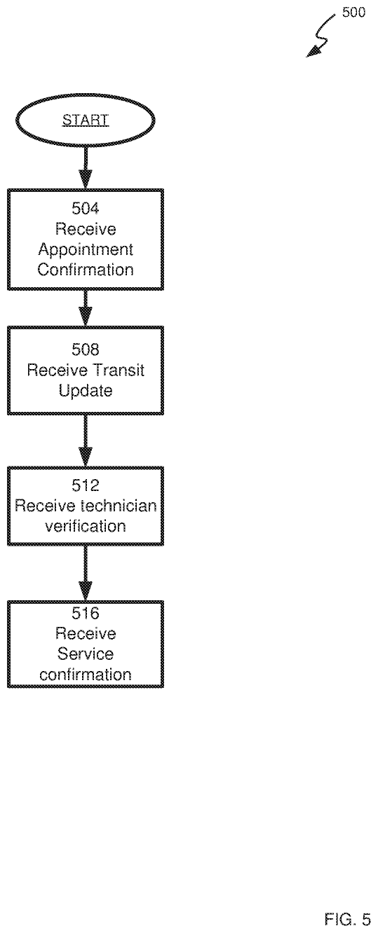

FIG. 5 shows a flowchart depicting a method of delivery of sample collection services indicated generally at 500. Method 500 can be implemented on a given patient device 112 within the framework of system 100. Thus, persons skilled in the art may choose to implement method 500 on one or more technician devices 112, system 100 or variants thereon, or with certain blocks omitted, performed in parallel or in a different order than shown. Method 500 can thus also be varied. However, for purposes of explanation, method 500 as per the flow chart of FIG. 5 and will be described in relation to its performance on patient device 112 within the framework of system 100 of FIG. 1.

Block 504 comprises receiving an appointment confirmation. As a given requisition is assigned to a given technician device 120, so to that assignment of that requisition can be confirmed in the form an appointment confirmation received on patient device 112. The confirmation can include an expected date, time of the appointment, an identification of the technician who will be performing the sample collection and what samples will be collected. A unique identifier for the requisition will be made available as part of the confirmation.

Block 508 comprises receiving appointment and/or transit updates. The updates are generated on the display of patient device 112. Such updates can be in real time. The updates can, if desired, include a map generated on device 112 showing the progress of a given vehicle 128 carrying a given technician towards the fixed location 114 of the patient. Real time updates as to the expected time of the appointment. Changes to the assignment of a given technician can also be generated if circumstances warrant assigning a different technician during performance of method 500.

Block 512 comprises receiving technician verification. Block 512 assumes that the assigned technician device 120 is now proximate to the patient device 112 and the requisition identification is confirmed between the relevant device 120 and relevant device 112. Such a confirmation can be effected using the above-described bar-code system or other means.

Block 516 comprises receiving service confirmation. Block 516 can include simply the results that confirm that the samples have been collected. Alternatively, or in addition, the service confirmation can include can a confirmation that the samples have been delivered to the laboratory. Alternatively, or in addition, the service confirmation can include can a confirmation that the test results have been delivered from the laboratory to the requisitioner. Alternatively, or in addition, the service confirmation can include can a copy of the test results themselves.

Notwithstanding any references to mobile laboratory technicians 119, patients 115, fixed locations 114, requisitioning entities 110 and/or laboratories 118 in these methods and elsewhere herein, it is to be understood that these are for illustrative purposes and convenience and that a person of skill in the art will appreciate that the load balancing and other technological implementations and advantages of the present specification relate to the load balancing that can be effected by machine engine 104 amongst the various computing resources of technician devices 120, requisitioner devices 108, patient devices 112 and/or laboratory devices 116, as well as potential carbon footprint mitigation of vehicles 128.

In another embodiment, matching engine 104 can receive test results from laboratory devices 116. Matching engine 104 can be configured to aggregate those test results for individual or demographic analytics. Matching engine 104 can also be configured to suggest follow up requisitions for individual patients based on the test results.

Databases 228 can be used to store different variables for system 100 that can be used to implement the various methods described herein. An example structure for certain databases 228 is shown in Table I.

TABLE 1

| | Variable | | |
|---|---|---|---|
| Database | ID | Name | Description |
| 228-1 | 1 | Requisitioner-Related | |
| | | Variables | |
| | 1-1 | requisitionerID | A unique identifier for |
| | | | each requisition. |
| | 1-2 | requistionerType | Doctor, Nurse, |
| | | | Employer, Insurance |
| | | | Underwriter or other |
| | | | type |
| | 1-3 | requistionerAuthority | What types of tests is |
| | | | the requisitioner |
| | | | permitted to request |
| 228-2 | 2 | Patient-Related Variables | |
| | 2-1 | patientID | A unique identifier for |
| | | | each patient. |
| | 2-2 | patientLocation | Current GPS |
| | | | coordinates of the |
| | | | patient. |
| | 2-3 | patientStatus | Indicates whether the |
| | | | patient has any |
| | | | relevant health |
| | | | conditions special |
| | | | needs during the |
| | | | administration of the |
| | | | test |
| | 2-4 | patientLocationtype | House, apartment |
| | | | building, office tower. |
| | 2-5 | patientRating | Patient's average |
| | | | rating based on past |
| | | | test. (e.g. ease of |
| | | | working with |
| | | | technician) |
| | 2-6 | patientLocationTransitStatus | Indicates variables |
| | | | such as access to on- |
| | | | premises parking, paid |
| | | | parking, public |
| | | | transportation access, |
| | | | taxi access. |
| | 2-7 | patientFeedback | Patient |
| | | | comments/ratings for |
| | | | a particular test and/or |
| | | | technicianID. |
| 228-3 | 3 | Technician-Related | |
| | | Variables | |
| | 3-1 | technicianID | A unique identifier for |
| | | | each technician. |
| | 3-2 | technicianLocation | Current GPS |
| | | | coordinates of the |
| | | | technician. |
| | 3-3 | technicianStatus | Indicates whether a |
| | | | technician is available, |
| | | | en route to a patient, |
| | | | waiting at a patient, |
| | | | administering tests to |
| | | | patient, en route to a |
| | | | laboratory, waiting at a |
| | | | laboratory |
| | 3-4 | technicianVehicleType | Car, bike, scooter, etc. |
| | 3-5 | technicianRating | Technician's average |
| | | | rating based on past |
| | | | test. |
| | 3-6 | technicianCapacity | The maximum number |
| | | | of tests a technician |
| | | | can carry/administer |
| | | | before visiting a |
| | | | laboratory. |
| | 3-7 | technicianETA | Estimated time of |
| | | | arrival at the |
| | | | laboratory or patient's |
| | | | location. |
| | 3-8 | technicianHistory | A record of past tests |
| | | | for each technician |
| | | | including patientIDs |
| 228-4 | 4 | Test-Related Variables | |
| | 4-1 | testID | A unique identifier for |
| | | | each test. |
| | 4-2 | testParameters | The types of tests to |
| | | | be administered |
| | 4-3 | testAdministrationTime | Time it takes/taken for |
| | | | technician to |
| | | | administer the test. |
| | 4-4 | testShelftime | Time from when test is |
| | | | collected that it must |
| | | | be delivered to the |
| | | | laboratory. |
| | 4-5 | testPatientAddress | Patient's delivery |
| | | | location. |
| 228-5 | 5 | Laboratory-Related | |
| | | Variables | |
| | 5-1 | laboratoryID | A unique identifier for |
| | | | the laboratory. |
| | 5-2 | laboratoryLocation | GPS coordinates of |
| | | | the laboratory. |
| | 5-3 | laboratoryParameters | The types of tests the |
| | | | laboratory can |
| | | | process. |
| | 5-4 | laboratoryWaitTime | Average time |
| | | | technicians spend |
| | | | waiting at the |
| | | | laboratory. This can |
| | | | vary based on |
| | | | laboratory efficiency, |
| | | | test complexity, etc. |
| | 5-5 | laboratoryHours | The times of day that |
| | | | the laboratory is open |
| | | | to receive tests from |
| | | | technicians. |
| 228-6 | 6 | Requisition | |
| | 6-1 | assignedTechnician | The technician |
| | | | assigned to a |
| | | | particular test. |
| | 6-2 | assignedTest | The test assigned to a |
| | | | particular technician. |
| | 6-3 | assignedPatient | The patientID of the |
| | | | patient who will |
| | | | receive the test |
| 228-7 | 7 | Operational Variables | |
| | 7-1 | trafficConditions | Real-time traffic |
| | | | conditions which might |
| | | | affect ETAs. |
| | 7-2 | weatherConditions | Weather can affect |
| | | | technician availability |
| | | | and ETAs, especially |
| | | | for those on bikes or |
| | | | scooters. |
| | 7-3 | peakHoursFlag | Indicates if it's a peak |
| | | | hour, which can affect |
| | | | technician availability |
| | | | and delivery times. |
| 228-8 | 8 | Matching Variables | |
| | 8-1 | matchingTimestamp | Time when the |
| | | | technician was |
| | | | matched to a |
| | | | requisitionID |
| | 8-2 | matchingRequisitionID | Any specific |
| | | | instructions from the |
| | | | requisitioner patient or |
| | | | laboratory regarding |
| | | | tests. |

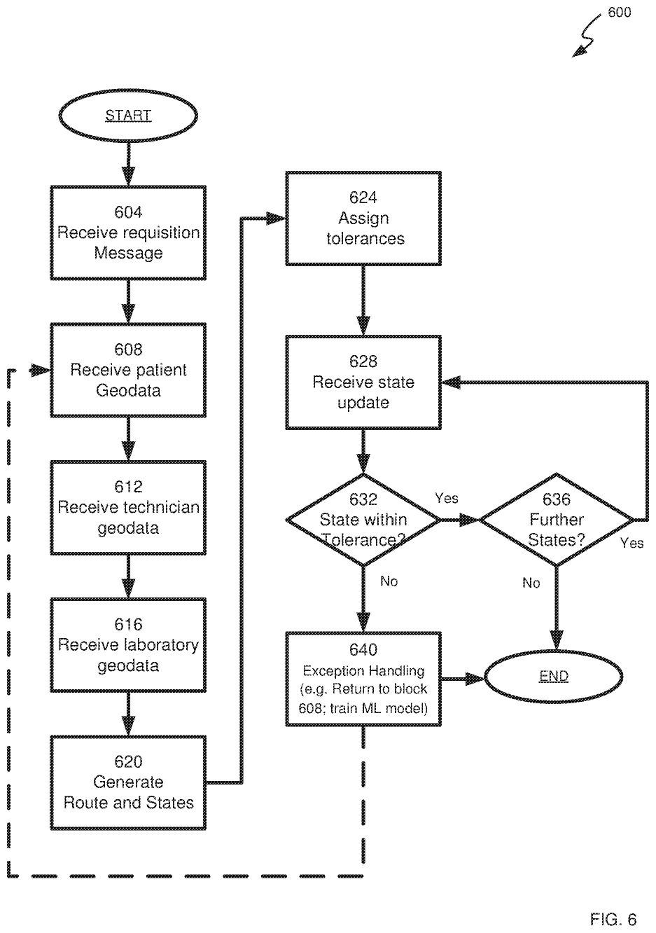

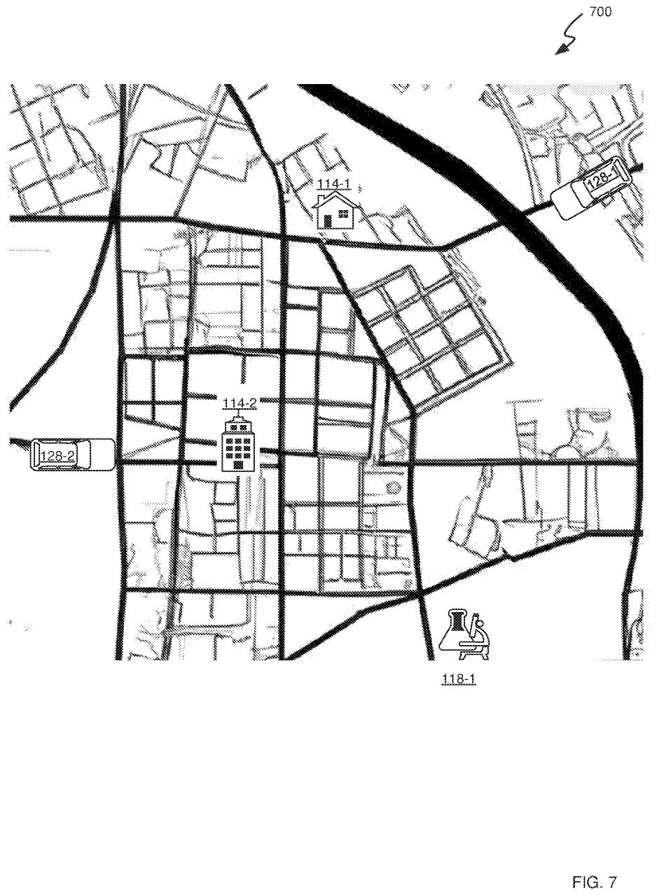

FIG. 6 shows a flowchart depicting another method for computational balancing of deployment of laboratory services indicated generally at 600. Method 600 can be implemented matching engine 104 within the framework of system 100. Thus, persons skilled in the art may choose to implement method 600 on matching engine 104, system 100 or variants thereon, or with certain blocks omitted, performed in parallel or in a different order than shown. Method 600 can thus also be varied. For purposes of explanation, method 600 as per the flow chart of FIG. 6 and will be described in relation system 100 with the assistance of a highly-simplified illustrative example in the form of the map 700 of FIG. 7 which shows two vehicles 128 and two fixed locations 114 and a laboratory 118-1.

Now, it is important to emphasize that the elements in FIG. 7 are graphical representations of a city map 700 including vehicles 128, locations 114 and laboratory 118, but that vehicles 128 are carrying respective technician device 120 and that locations 114 are housing respective patient devices 112 and that laboratory 118 is housing a respective laboratory devices 116. FIG. 7 is thus an assistive visual aid to help a person skilled in the art to appreciate technical performance of method 600 on engine 104.

Block 604 comprises receiving a requisition message. As noted earlier, the requisition message can originate from, for example, requisitioner device 108-1. In our example of FIG. 7, we will further assume that the requisition is for patient 115-1 and its for a cholesterol blood test.

Block 608 comprises receiving patient geodata. Matching engine 104 can thus connect to devices 112 and ascertain the location of device 112-1 on map 700 and identify the global positioning system (GPS) coordinates of fixed location 114-1.

Block 612 comprises receiving technician geodata. Matching engine 104 can thus connect to technician devices 120 and identify the current location of vehicle 128-1 respective to technician device 120-1 and vehicle 128-2 respective to technician device 120-2.

Block 616 comprises receiving laboratory geolocation data. Matching engine 104 can thus connect to laboratory device 116-1 and ascertain the location of device 116-1 on map 700 and identify the global positioning system (GPS) coordinates of laboratory 118-1.

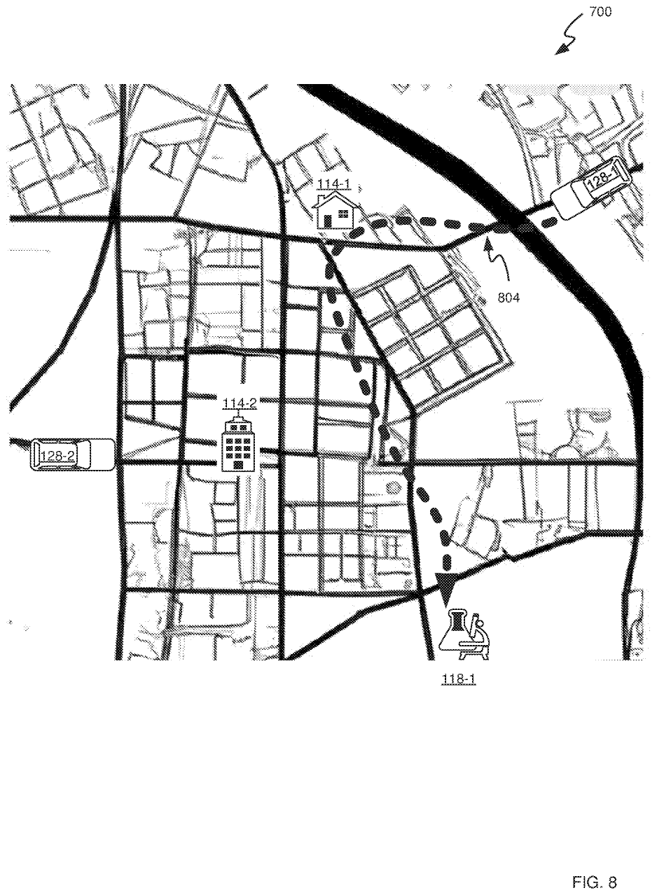

Block 620 comprises generating a route and the associated states. The route can include a geospatial electronic routemap for each technician device 120. The computation of the route can be based on all of the variables in databases 228, considering, by way of non-limiting example, proximities, traffic conditions, test times, travel times, availabilities, in order to optimize the fastest fulfillment of the requisition in terms of test sample collection and delivery to laboratory 118-1. Any and/or all of the variables in databases 228 may be considered as appropriate. FIG. 8 shows a first example performance of block 620, which generates route 804 where technician device 120-1 (within vehicle 128-1) is scheduled to rendezvous GPS coordinates with patient device 112-1 at location 114-1 at a given time. Such a rendezvous may be considered an appointment commitment at a given time for a mobile laboratory technician 119 with a given patient 115. Based on variable 4-3 “testAdministrationTime” of database 228-4, the route 804 can include a period of time that the rendezvous would last. Route 804 thus also includes an accommodation for travel time from location 114-1 to laboratory 118-1. The reference to variable 4-3 is just an example, as a person skilled in the art reviewing Table I can appreciate all of the variables that can be incorporated innt route 804. Block 620 thus also contemplates assigning various states to the route 804, including:
1) travel from the initial location of vehicle 128-1 to location 114-1;
2) park a vehicle at location 114-1,
3) administration the test in the requisition,
4) travel from location 114-1 to laboratory 118-1; and,
5) wait at laboratory 118-1.

These are five examples of predicted electronic states, but different states can be conceived.

Block 624 comprises assigning tolerances to the route from block 620. The tolerances can be based on expected ranges of times, or time-frames, for each of the predicted states. As a salient example, the test sample collected at location 114-1 may only have a certain “shelf life” (variable 4-4 from database 228-4, “testShelftime”), and thus the tolerances at block 624 for state 4) “4) travel from location 114-1 to laboratory 118-1 and 5) wait at laboratory 118-1” should not exceed the “testShelftime” according to the tolerance established at block 624.

Another tolerance can include states 1) travel from the initial location of vehicle 128-1 to location 114-1 and 2) park a vehicle at location 114-1, being the time-window that a patient device 112-1 should be expected to remain at location 114-1 to be available for the administration of the test.

Another tolerance can be associated with state 3) administration the test in the requisition. This tolerance can be a range applied to variable 4-3 “testAdministrationTime” that may also consider variables such as database 228-3 including variable 3-5 “technicianRating” and/or variable 3-8 “technicianHistory” and/or variable 2-5 “patientRating”. Other variables that can associated with the tolerances will occur to those of skill in the art.

Block 628 comprises receiving an update of the current state. This portion of method 600 (which can stand along from method 600 altogether) monitors the fulfillment of route from block 620. The updates can consider which state the route is in and the time elapsed in that state. Block 628 can thus include monitoring real-time electronic state updates of mobile devices such as technician devices 120 and/or patient devices 112 and/or laboratory devices 116 and compare with the predicted states from block 620.

To elaborate, block 632 can comprise determining if the current state is within the tolerance from block 624. A “Yes” determination leads to block 636, and if there are further states then method 600 cycles back to block 628. Assuming the route from block 620 is being fulfilled within the tolerances, then method 600 will eventually terminate avoiding the exception handling routine at block 640. In this ideal scenario for route 804, matching engine 104 will monitor the travel of device 120-1, its rendezvous with device 112-1, the time device 120-1 remains near device 112-1, the time device 120-1 takes to travel to laboratory device 116-1 and/or laboratory 118-1, and all of these times will be within the tolerances assigned at block 624. It should be noted that state updates can optionally include receipt of periodic manual input at device 120-1, device 112-1 and laboratory device 116-1 from respective the technician, patient and laboratory technician with any unstructured notes and/or structured annotations such as indicating “complete”, entering ratings or the like.

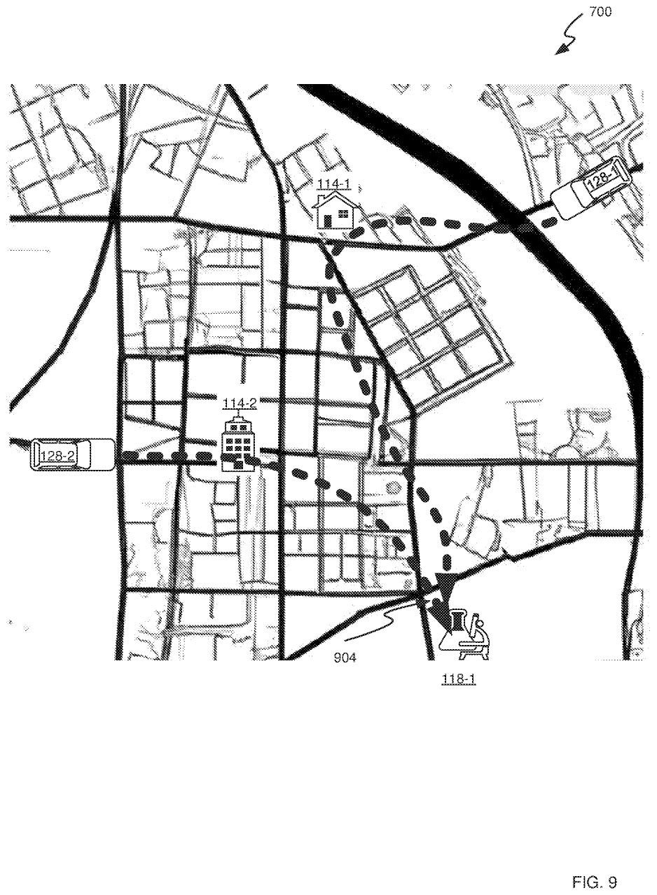

FIG. 9 shows another idealized secondary performance of method 600 in relation to a second requisition for patient 115-2, who is using device 112-2 at location 114-2. Given proximity of device 120-2 in vehicle 128-2, technician 119-2 is assigned to patient 115-2 and route 904 is generated. Again, in an ideal scenario, method 600 cycles from block 628, block 632 and block 636 until all states are satisfied at method 600 advances from block 636 to “End”, avoiding exception handling block 640 altogether.

Table II shows these two idealized examples including example timing relevant variables from Table I.

TABLE II

| | | Technician | Technician | |
|---|---|---|---|---|
| Time | Event/Action | 119-1 | 119-2 | Relevant Variables |
| 10:00 | Test 1 | Notified, En | — | uhtxlvlwlr [symbol] huLG, whvwLG, |
| | requisitioned for | route to Patient | | sdwlh [symbol] wLG, whfk [symbol] lfld [symbol] LG, |
| | Patient 115-1 | 115-1 | | whfk [symbol] lfld [symbol] Orfdwlr [symbol] , |
| | | | | whfk [symbol] lfld [symbol] Vwdwxv |
| 10:10 | Test 2 | Administering | Notified, En | uhtxlvlwlr [symbol] h [symbol] LG, whvwLG, |
| | requisitioned for | Test 1 | route to Patient | sdwlh [symbol] wLG, whfk [symbol] lfld [symbol] LG, |
| | Patient 115-2 | | 115-2 | whfk [symbol] lfld [symbol] Orfdwlr [symbol] , |
| | | | | whf [symbol] lfld [symbol] Vwdwxv |
| 10:20 | Technician A | En route to | Administering | whfk [symbol] lfld [symbol] Vwdwxv, whvwVkho [symbol] wlp [symbol] , |
| | finishes with | Laboratory | Test 2 | whvwD [symbol] pl [symbol] lvwudwlr [symbol] Wlph, |
| | Patient 115-1 | 118-1 | | whfk [symbol] lfld [symbol] HWD, |
| | | | | cderudwru\|Orfdwl [symbol] |
| 10:30 | Technician B | Waiting at | En route to | whfk [symbol] lfld [symbol] Vwdwxv, whvwVkhoiwlph, |
| | finishes with | laboratory | Laboratory | whvwD [symbol] l [symbol] lvwudwlr [symbol] Wlph, |
| | Patient 115-2 | 118-1 | 118-1 | whfk [symbol] lfld [symbol] HWD, |
| | | | | oderudwru\|Orfdwlr [symbol] |
| 10:40 | Technician A | Available | Waiting at | whfk [symbol] lfld [symbol] Vwdwxv, |
| | delivers Test 1 to | | laboratory | oderudwru\|ZdlwWlph |
| | Laboratory 118-1 | | 118-1 | |
| 10:50 | Technician B | Available | Available | whfk [symbol] lfld [symbol] Vwdwx [symbol] , |
| | delivers Test 2 to | | | oderudwru\|ZdlwWlph |
| | Laboratory 118-1 | | | |

[symbol]

indicates data missing or illegible when filed

Over time, machine learning can be applied to several historic performances of method 600 to achieve the idealized routes such as example route 804 and route 904. It is contemplated, however, that from time to time uncontrollable events will lead to “No” determinations at block 632, thereby leading to exception handling at block 604. As a very simple example, we can refer to route 804 and route 904 which we can assume happen on the same day within geographic proximity as per map 700. If we make an assumption that, for whatever reason, vehicle 128-1 does not arrive at location 114-1 within the allotted tolerance at block 624 for the state “1) travel from the initial location of vehicle 128-1 to location 114-1”, then a “no” determination will occur at block 632 and method 600 will advance to block 640 for exception handling.

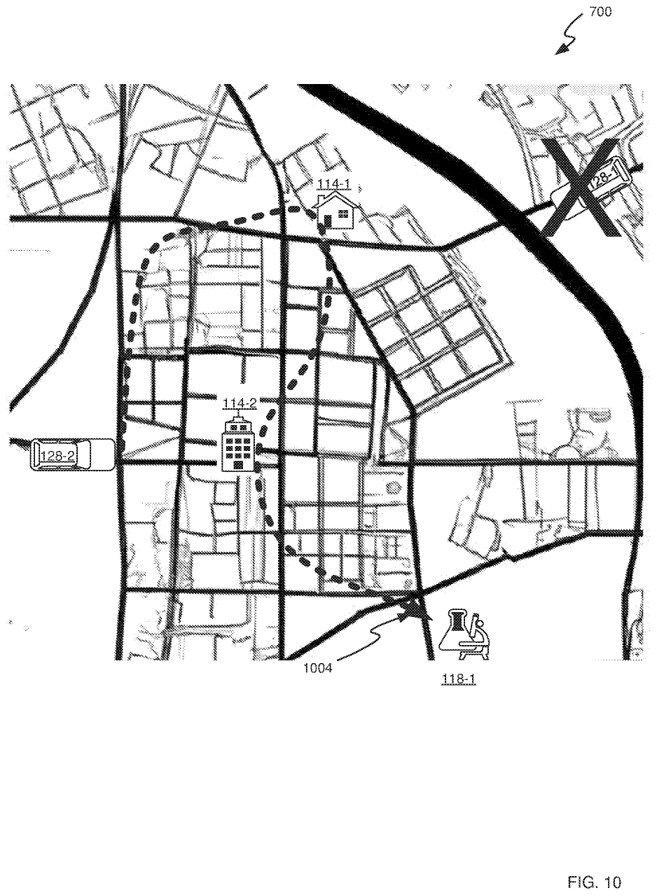

The nature of the exception handling is not particularly limited and can include, for example, invocation (or reinvocation) of method 300, method 400 or method 500 or appropriate variations thereon or combinations thereof. In very general terms, block 640 can include adjusting the routemap of block 620 for one or more of the mobile devices such as technician devices 120 if any real-time state detected at block 628 diverges from the tolerances as per block 632. In a present example embodiment, block 640 includes a return to block 608 where the requisition message previously received at block 604 is reprocessed from block 608 onwards. According to our example, if vehicle 128-1 (and by extension, technician 119-1) become unavailable then matching engine 104 can reassess system 100 and reassign the requisition. Table III and FIG. 10, shows just such an example where Route 1004 is generated for technician 119-2 to fulfill both requisitions.

TABLE III

| | | Technician | Technician | |
|---|---|---|---|---|
| Time | Event/Action | 119-1 | 119-2 | Relevant Variables |
| 10:00 | Test 1 | Notified, En | — | uhtxlvlwlr [symbol] huLG, whvwLG, |
| | requisitioned for | route to | | sdwlh [symbol] wLG, whfk [symbol] lfld [symbol] LG, |
| | Patient 115-1 | Patient 115-1 | | whfk [symbol] lfld [symbol] Orfdwlr [symbol] , |
| | | (Route 804 | | whfk [symbol] lfld [symbol] Vwdwxv |
| | | created) | | |
| 10:05 | Technician 119-1 | Unavailable | Notified, En route | whfk [symbol] lfld [symbol] Vwdwxv |
| | becomes | (Route 804 | to Patient 115-1 | |
| | unavailable | deleted) | | |
| 10:10 | Test 2 | — | Notified of Test 2, | uhtxlvlwlr [symbol] huLG, whvwLG, |
| | requisitioned for | | Continues to | sdwlh [symbol] wLG, whfk [symbol] lfld [symbol] LG |
| | Patient 115-2 | | Patient 115-1 | |
| 10:20 | Technician 119-2 | — | En route to | whfk [symbol] lfld [symbol] Vwdwxv, |
| | finishes with | | Patient 119-2 | whvwD [symbol] pl [symbol] lvwudwlr [symbol] Wlph, |
| | Patient 115-1 | | | whfk [symbol] lfld [symbol] HW [symbol] |
| 10:30 | Technician 119-2 | — | Administering | whfk [symbol] lfld [symbol] Vwdwxv, |
| | arrives at Patient | | Test 119-2 | whvwD [symbol] pl [symbol] lvwudwlr [symbol] Wlph |
| | 115-2 | | | |
| 10:40 | Technician 119-2 | — | En route to | whfk [symbol] lfld [symbol] Vwdwxv, |
| | finishes with | | Laboratory 118-1 | whvwVkho [symbol] wlph, whfk [symbol] lfld [symbol] HWD, |
| | Patient 115-2 | | | oderudwru\|Orfdwlr [symbol] |
| 10:50 | Technician 119- | — | Available | whfk [symbol] lfld [symbol] Vwdwxv, |
| | 2delivers both | | | oderudwru\|ZdlwWlph |
| | tests to Laboratory | | | |
| | 118-1 | | | |

[symbol]

indicates data missing or illegible when filed

Accordingly, in this variant, one or more of the applications 224 may include machine learning or artificial intelligence with any desired related machine learning deep-learning based algorithms and/or neural networks, and the like, which are trained to improve the machine learning functions at block 640. Such improvement can be used during subsequent performances of method 300, method 400, method 500 and/or method 600 and/or combinations thereof, and/or variations thereon, in order to reduce future exception handling scenarios at block 640. The machine learning applications 224 may be operated by the processor 208 in a training mode to train the machine learning and/or deep-learning based algorithms and/or neural networks of the machine learning applications 224 in accordance with the teachings herein.

The one or more machine-learning algorithms and/or deep learning algorithms and/or neural networks of the machine learning applications 224 may include, but are not limited to: a generalized linear regression algorithm; a random forest algorithm; a support vector machine algorithm; a gradient boosting regression algorithm; a decision tree algorithm; a generalized additive model; neural network algorithms; deep learning algorithms; evolutionary programming algorithms; Bayesian inference algorithms; reinforcement learning algorithms, and the like. However, generalized linear regression algorithms, random forest algorithms, support vector machine algorithms, gradient boosting regression algorithms, decision tree algorithms, generalized additive models, and the like may be preferred over neural network algorithms, deep learning algorithms, evolutionary programming algorithms, and the like.

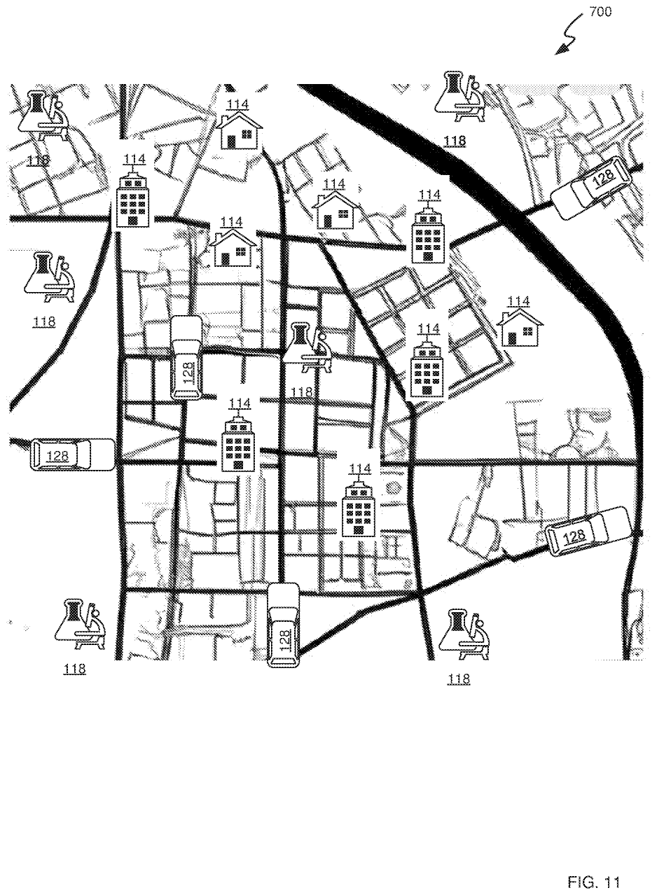

It is to be emphasized that the example route 804, route 904 and route 1004 and the accompanying Table II and Table III are highly simplified for illustrative purposes. A person of skill in the art will appreciate how system 100 and method 300, method 400, method 500 and method 600 can be massively scaled across a defined geographic region and/or across the globe. FIG. 11 attempts to give a visual sense of the beginnings of such a scaling capability, as a plurality of locations 114, vehicles 128 and laboratories 128 are shown dispersed across only a portion of an urban area, all of which represent potential locations of patient devices 112 with patient requisitions, technician devices 120 and laboratory devices 116 whose computational efficiencies can be managed using the present specification while also managing carbon footprints of vehicles 128. While requisitions and testing are typically geofenced within a particular urban area, overlaps can be contemplated and machine learning from one geographic area may be appropriately applied to another geographic area as part of scaling. Again FIG. 11 is only the beginning of such scaling, as hundreds, thousands, tens of thousands and more requisitioner devices 108, patient devices 112, laboratory devices 116, laboratories 118 and technician devices 120 can be managed by matching engine 104 (or scaled version or versions thereof), well beyond any scaling that can be managed by human, especially when considering the machine learning optimization that can occur.

It is also to be understood that routes can be generated to reduce carbon footprints of vehicles 129, reduce the amount of time that a patient 115 waits at a location 114, reduce the amount of time to deliver test samples to laboratories 118, reduce the number of mobile laboratory technicians 119 and/or determine a best match between mobile laboratory technicians 119 and patients 115. Block 620 can be configured to optimize for one or more of these reductions and/or any other desired criteria. It should also be noted that the example in Table III is also ripe for massive scaling, as dozens or hundreds of routes may be initially generated but also dynamically updated in real time, again with the criteria of reducing carbon footprints, reducing the amount of time that a patient 115 waits, reducing the amount of time to deliver test samples to laboratories 118. One of the most complex variables to manage is the shelf life of a test sample, such that if a given mobile laboratory technicians 119 is faced with multiple updates to their routes during a shift, matching engine 104 can also make such route adjustments while noting whether shelf life of test samples will remain within accepted tolerances-real time calculations that are cannot be performed by a human within the confines of the practical realities of meeting patients 115 within acceptable time windows, closing hours of laboratory 118 and shelf life of test samples as the routes are being fulfilled.

System 100 can also be configured so that the tolerances at block 624 accumulate for every additional test that is scheduled for mobile laboratory technicians 119 before delivery of the test samples to laboratory 118. Indeed, which laboratory 118 is chosen can also be dynamically updated as part of block 640.

The tolerances at block 624 can also, in extraordinary circumstances, including considering the possibility of “acceptable losses”. To elaborate, if a particular route is already full for a technician, but an “urgent” requisition is received, (e.g. a potentially life threatening situation associated with an urgent need to fulfill the tests from a requisition to make a medical decision) the tolerances at block 624 may permit for spoilage of one or more test samples that is non-urgent, such that the test collection is rescheduled automatically for another day.

In view of the above it will now be apparent that variants, subsets, and/or combinations of the embodiments and teachings are contemplated. For example, the foregoing has been discussed in relation to the deliver of laboratory services, it will now be understood that the above-described embodiments can be modified more broadly to the delivery of other medical services.

In another variant, a given technician 119 can simply be routed to a given location 114, and only upon arrival is the specific requisition actually received at the relevant technician device 120.

With all the collected patient activity, including location and time when registered, location and time they uploaded a requisition, and app activity system 100 can generate predictive heat maps for where patient may request service in the future. These predictions can augment the ability to interact with the patient based on actual tests requested, and system 100 can interact with a patient instructing them to follow any preparation needed for the collection. For example, if requisition with fasting sugar test is uploaded, then system 100 can decides to generate demand at a certain hour of the day on a certain day of the week, then system 100 will start engaging the patient suggest when they should fast and when they should schedule the test.

System 100 can also accommodate geofencing capability to match demand for laboratory technicians with the actual supply of laboratory technicians using the predictive nature of system 100, the patient engagement reporting and the ability to create heat maps where the laboratory technician can be instructed to be in certain areas ahead of the dally demand.

The route generation of block 316 and/or block 620 and/or tolerances from block 624 can include configuring engine 104 determines the optimal set of mobile laboratory technicians 119 based on a target minimum number of mobile laboratory technicians 119 currently available using the geodata from technician devices 120.

Another feature establishes criteria for adjusting schedules, such as a return from block 320 to block 304 and/or block 640. These criteria can include one or more of responding to potential sample degradation due to factors like traffic delays by sending the mobile laboratory technicians 119 to the laboratory 118. The criteria can take into account various factors while scheduling, such as: mitigating missed appointments for patients 115; mitigating sample degradation; mitigating the number of mobile laboratory technicians 119, attempting to maximize the number of appointments with patient 115 per mobile laboratory technician 119.

The criteria can include real-time monitoring of technician availability and reallocating them to ensure patient appointments are met and sample integrity is maintained.

The criteria can include evaluating if schedule changes can be made without resulting in more missed appointments for patients.

As discussed, if there is a risk of samples degrading, system 100 can re-optimize the schedules for each mobile laboratory technician 119 and patient 115 and/or laboratory 118. Moreover, machine learning can be utilized to assess whether a predicted schedule (at block 620) or schedule change resulting from block 640 increased or decreased tolerance violations from block 632. Based on this analysis, the criteria for establishing and/or rebalancing the scheduled route at block 620 can be updated.

An aspect of the specification provides a matching and scheduling engine for deploying phlebotomy services including a processor configured to implement a plurality of programming instructions: receiving a plurality of electronic data files representing phlebotomy requisitions; requisitions are structured in an available list receiving a plurality of patient electronic profiles corresponding to a respective requisition file; each patient electronic profile including: a patient location; a patient preferred service time; a sample collection-profile for the requisition; a patient ranking-profile; receiving a plurality of laboratory electronic profiles: each laboratory electronic profile including: a laboratory location; a laboratory capacity for each sample collection-profile; receiving a plurality of technician electronic profiles each technician electronic profile including: a technician origin; a technician availability window; a technician qualification; a technician geofence; a technician ranking-profile; determining a service-delivery schedule for each technician based on the patient electronic profiles, laboratory electronic profiles and technician electronic profiles such that: each patient receives the service proximate to the preferred service-time; an optimal set of technicians are matched for deployment to each patient-location; an optimal set of laboratories are matched with the set of technicians according to laboratory location and laboratory capacity.

A person skilled in the art will now appreciate that the computational load balancing of the teachings herein can have the incidental benefit of improving the speed, efficiency and accuracy with which laboratory tests are requisitioned, collected, processed, delivered and actioned. A technological benefit can be based on the dynamic route generation which effects load balancing by matching the processing resources of the devices that track the collection of samples, and the resources of the devices the aggregate the results and deliver the results to patients. The present teachings can be used to load balance the processing of test results across laboratory devices 116 and thereby improve computational and network efficiency of system 100. To elaborate, in a prior art scenario without the present teachings, one laboratory device 116 may be tasked with processing a plurality of tests while other devices 116 remain idle. The present teachings, on the other hand, can achieve a balancing of processing samples across a plurality of laboratory devices 116.

Another example of a technical advantage from the present specification is illustrated by comparing the initial state in FIG. 7 with the example in FIG. 10. To elaborate, system 100 can be configured to determine that only technician device 120-2 is required to service both requisitions for patient 115-1 and patient 115-2, and thus skipping over the example of FIG. 8 and FIG. 9 altogether, and thereby only deploying device 120-2 and “sidelining” device 120-1 altogether. The result conserves computational and network resources associated with device 120-1, without overwhelming the resources of device 120-2 and indeed making use of extra computational capacity of device 120-2. Another technical advantage according to this example is a reduction of overall carbon footprint for system 100 by deploying only a single vehicle 128-2 and “sidelining” vehicle 128-1.

Now, these two examples can also be combined as combined criteria, considering the need to load balance resources of laboratory device 116 while also reducing carbon footprints of vehicles 128 and computational resources used by devices 120. It is to be understood that these two criteria, either individually or combined, are non-limiting examples of potential implementations that can be used to reduce and/or optimize and/or load-balance computational and/or network load in system 100 and/or reduce carbon footprints of vehicles 128. Notably, any such criteria can also consider the “testShelftime” of any plurality of samples.

Prior art systems are disjointed, with unique applications for the requisitioner, the patient, and the laboratory leading to inefficient use of computing and network resources. Other benefits will now occur to those skilled in the art.

It should be recognized that features and aspects of the various examples provided above can be combined into further examples that also fall within the scope of the present disclosure. In addition, the figures are not to scale and may have size and shape exaggerated for illustrative purposes.

1. A matching engine for computational load balancing comprising a network interface, a processor and a memory for storing programming instructions for configuring the processor to:
receive at least one laboratory requisition electronic message;
receive at least one primary geolocation dataset of at least one target location of at least one primary electronic device respective to each message;
receive at least one mobile geolocation dataset of at least one location of at least one mobile electronic device within a geographic range proximal to at least one primary electronic devices of the primary geolocation dataset;
receive at least one secondary geolocation dataset of at least one final location of at least one computation device within a geographic range proximal to the primary geolocation dataset;
generate a geospatial electronic routemap for each mobile electronic device including travel from i) an origin within the mobile geolocation dataset, to ii) the at least one target location of one or more primary electronic devices, and then to iii) the at least one final location of the at least one computation device;
generate expected electronic states for each stage of the electronic routemap, the expected electronic states including expected times and expected time-frames;
assign tolerances to the expected electronic states, the tolerances based on the expected time-frames for each of the expected electronic states;
receive real-time updates of actual electronic states of each mobile electronic device, each update including a current state and time elapsed in the current state and compare the actual electronic states to the expected electronic states; and,
update the geographic electronic routemap for at least one of the mobile electronic devices if the actual electronic states are outside the tolerances of the expected electronic states.

2. The matching engine of claim 1, wherein the tolerances are based at least in part on a time-frame for potential sample degradation during travel from the target location of a respective primary device to an assigned computation device location.

3. The matching engine of claim 1, wherein the tolerances are based at least in part on arrival time windows at the target locations of the primary devices.

4. The matching engine of claim 1 wherein the tolerances are based at least in part on reducing the deployment of mobile devices of laboratory technicians.

5. The matching engine of claim 1 wherein the tolerances are based at least in part on reducing a carbon footprint of vehicles associated with the mobile devices.

6. The matching engine of claim 1 wherein the time-frame tolerances are based at least in part maximizing a number of primary devices assigned per mobile device.

7. The matching engine of claim 1, wherein criteria for adjusting include real-time monitoring of technician mobile device availability and reallocation of technician mobile device to uphold primary device appointment commitments and maintain sample integrity.

8. The matching engine of claim 1, wherein the adjusting includes assessing if scheduling adjustments can be implemented without increasing the likelihood of missed primary device appointment commitments.

9. The matching engine of claim 1, further comprising machine learning to evaluate the impact of scheduled routes or changes to said routes on tolerance violations, and adjust criteria for the generating and the adjusting based on the evaluation results.

10. The matching engine of claim 1 wherein the expected electronic states further include one or more of i) parking a vehicle; ii) administering the test in the requisition; and iii) waiting for processing of test results.

11. A method for computational load balancing by a matching engine comprising:
receiving at least one laboratory requisition message;
receiving primary geolocation data of target locations of primary devices related to each message;
receiving mobile geolocation data of mobile devices proximal to the primary devices;
receiving secondary geolocation data of computation device locations near the primary locations;
generating a geospatial routemap for each mobile device respective to each said message including travel from: i) a starting point in the mobile geolocation data, to ii) target locations of primary devices, and then to iii) computation device locations;
generating predicted electronic states for each routemap stage, the predicted electronic states including predicted times and predicted time-frames;
assigning tolerances for each of the states, the tolerances based on the predicted time-frames for each of the predicted electronic states;
monitoring real-time electronic state updates of mobile devices, each update including a current state and time elapsed in the current state, and comparing the updates with the predicted electronic states; and,
adjusting the routemap for one or more of the mobile devices if any real-time state diverges from the tolerances.

12. The method of claim 11, wherein the tolerances are based at least in part on a time-frame for potential sample degradation during travel from the target location of a respective primary device to an assigned computation device location.

13. The method of claim 11, wherein the tolerances are based at least in part on arrival time windows at the target locations of the primary devices.

14. The method of claim 11 wherein the tolerances are based at least in part on reducing the deployment of mobile devices of laboratory technicians.

15. The method of claim 11 wherein the tolerances are based at least in part on reducing a carbon footprint of vehicles associated with the mobile devices.

16. The method of claim 11 wherein the time-frame tolerances are based at least in part maximizing a number of primary devices assigned per mobile device.

17. The method of any claim 11, wherein criteria for adjusting include real-time monitoring of technician mobile device availability and reallocation of technician mobile device to uphold primary device appointment commitments and maintain sample integrity.

18. The method of claim 11, wherein the adjusting includes assessing if scheduling adjustments can be implemented without increasing the likelihood of missed primary device appointment commitments.

19. The method of claim 11, further comprising machine learning to evaluate the impact of scheduled routes or changes to said routes on tolerance violations, and adjust criteria for the generating and the adjusting based on the evaluation results.

20. The method of claim 11 wherein the predicted electronic states further include one or more of i) parking a vehicle; ii) administering the test in the requisition; and iii) waiting for processing of test results.
